(19) United States
(12) Patent Application Publication
(10) Pub. No.: US 2016/0193226 A1
(43) Pub. Date: Jul. 7, 2016

(54) Composition for Increasing Libido

(71) Applicant: Ludovic Audesson, New York, NY (US)

(72) Inventor: Ludovic Audesson, New York, NY (US)

(21) Appl. No.: 14/989,366

(22) Filed: Jan. 6, 2016

Related U.S. Application Data
(60) Provisional application No. 62/100,706, filed on Jan. 7, 2015.

(51) Int. Cl. A61K 31/568; A61K 9/00; A61K 35/12; A61K 31/728; A61K 31/565

(52) U.S. Cl. CPC A61K 31/568; A61K 31/728; A61K 31/565; A61K 35/12; A61K 9/0014

(57) ABSTRACT

A composition for improving a person's libido and rejuvenating the skin. The composition includes effective amounts of a human sex hormone, such as testosterone or estrogen so as to increase the user's hormone levels. The composition further includes hyaluronic acid that serves to tighten or moisturize the user's skin, particularly the skin near or around a sexual organ. Some embodiments further include a pheromone for providing a relaxing effect on the user and to help improve the user's mood. In some embodiments, the composition further includes stem cells for promoting repair and rejuvenation of damaged tissue in sexual organs. The composition is preferably provided in a cream so that it is adapted for topical application directly to the user's skin.

### CROSS REFERENCE TO RELATED APPLICATION

This application claims the benefit of U.S. Provisional Application No. 62/100,706 filed on Jan. 7, 2015. The above identified patent application is herein incorporated by reference in its entirety to provide continuity of disclosure.

### BACKGROUND OF THE INVENTION

### Field of the Invention

The present invention relates to sexual enhancement compositions. More specifically, the present invention provides a composition for increasing a user's libido comprising effective amounts of human sex hormones and hyaluronic acid.

An individual's libido or sex drive is influenced by many factors including social, physiological, and biological factors. Biological factors are strongly correlated with a person's sex drive. Particularly, sex hormones such as testosterone, estrogen, and progesterone influence a person's sex drive. A person having low hormone levels may experience a decreased sex drive and may have sexual dysfunction. A person's sex drive generally peaks in the teenage years and declines gradually as the person ages into their forties and beyond.

Physiological factors such as a person's mental health issues, including anxiety or depression, may influence a person's sex drive. Further, a person's sex drive may be affected by their personality, stress, sleep patterns, and lifestyle. Certain medical conditions or medications may also negatively affect a person's sex drive, causing decreased arousal or sensitivity. Thus, many factors contribute to a person's sexual drive and sexual drive may vary greatly among different people.

Another factor that has a significant impact on a person's libido is their age. Generally, males reach the peak of their sex drive in their teenage years and their sex drive gradually drops over the course of their life. Similarly, a female's sex drive peaks in her twenties or thirties and slowly drops thereafter. Thus, as a person ages, their libido generally drops, particularly for persons that are forty or older.

Compositions and methods have been disclosed in the prior art that relate improving sexual performance, such as U.S. Published Patent Application Number 2011/0086923, U.S. Pat. No. 5,698,589, U.S. Published Patent Application Number 2012/0061487, U.S. Patent Number 2011/0201579, U.S. Pat. No. 5,059,603, and U.S. Published Patent Application Number 2011/0151028.

These prior art devices have several known drawbacks. The compositions in the prior art relate to sexual dysfunction but do not serve to increase the user's libido. Further, such compositions do not serve to increase the user's hormone levels in order to increase the user's libido. Thus, an improved composition for helping to increase libido is desired.

In light of the compositions disclosed in the prior art, it is submitted that the present invention substantially diverges from the prior art and consequently it is clear that there is a need in the art for an improved composition for improving sexual drive. In this regard the instant invention substantially fulfills these needs.

### SUMMARY OF THE INVENTION

In view of the foregoing disadvantages inherent in the known types of compositions for improving sexual drive now present in the prior art, the present invention provides a new composition wherein the same can be utilized for providing convenience for the user when increasing a person's sexual drive.

It is therefore an object of the present invention to provide a new and improved composition comprising human sex hormones adapted to increase hormone levels, increasing sexual drive.

It is another object of the present invention to provide a new and improved composition comprising hyaluronic acid for use in treating dry skin and providing a moisturizing effect.

Another object of the present invention is to provide a new and improved composition comprising a pheromone for increasing attraction or for triggering sexual arousal.

Yet another object of the present invention is to provide a composition that is preferably in the form of a cream for topical application to a user's sexual organs.

### DETAILED DESCRIPTION OF THE INVENTION

For the purposes of presenting a brief and clear description of the present invention, the preferred embodiment will be discussed as used for increasing a user's libido.

A composition for increasing a user's libido is provided. The composition primarily comprises an effective amount of one or more sex hormones, hyaluronic acid, and one or more pheromones. The composition is preferably in the form of a cream so that it can be applied topically. The cream is adapted to be applied directly to the user's skin and/or sexual organs. The composition serves to tighten and moisturize the skin of the user's sexual organs. Further, the hormones of the composition are absorbed into the user's body and cause an increase in the user's libido.

In a first embodiment, the sexual hormone is testosterone, and the composition is adapted for use by men. Testosterone levels are positively correlated with a male's libido or sex drive. Low testosterone levels can result in a low sexual drive and may also cause erectile dysfunction. As a male grows older, his testosterone levels decline, which results in a declined libido. As a result, testosterone therapy can be used to increase a male's testosterone levels and therefore increase his sexual drive. Further, increase testosterone levels help to prevent erectile dysfunction and related conditions.

In an alternate embodiment, the sexual hormone is estrogen, and the composition is adapted for use by females. In females, estrogen levels are positively correlated with sexual drive. Women having lower estrogen levels are more likely to have a decreased sexual drive. Further, low estrogen levels may result in vaginal dryness which may make sexual intercourse painful. Low estrogen also contributes to unstable moods and sleep patterns, which may also inhibit the woman's desire to engage in sexual activity. This is more common in older women, such as women who are in their fifties or older. As a result, providing estrogen to women having low estrogen levels and a low sex drive may help to increase or improve the woman's sex drive. Estrogen creams can be applied topically to increase vaginal lubrication and sexual arousal.

The composition further comprises hyaluronic acid. Sex hormones alone may be ineffective in increasing the user's libido and the hormones do not repair or treat the user's sexual organs. This is particularly important for females who may suffer from vaginal dryness as the result of low hormone levels, wherein the dryness may not be resolved by hormone treatment alone. Hyaluronic acid is found in body tissues such as the skin and cartilage. Hyaluronic acid is a major component of human skin and is involved in the tissue repair process. Hyaluronic acid has been used to treat dry skin and is able to be applied topically to an area of dry skin. As a result, hyaluronic acid is used in skin-care products in order to treat dry skin. In alternate embodiments, a moisturizing agent is included in the composition so as to help to treat dry skin.

The composition further comprises one or more pheromones. Pheromones, as used herein, refers to any chemical or substance that can help the user to relax or relieve stress and that can provide an increase in the person's mood. The pheromone is adapted to provide a pleasant scent to help relax the user and improve his or her mood. In one embodiment, the pheromone is jasmine extract. In this way, application of the composition including a pheromone will help to improve the user's mood and help the user to relax, which may further augment the user's sex drive. Any of various pheromones may be used in the composition of the present invention, and it is not desired to limit the specific type of pheromone utilized.

In some embodiments, the composition further comprises an effective amount of stem cells. Stem cells serve to repair and rejuvenate the user's sexual organs. Stem cells can be used to stimulate the growth of human tissue. An effective concentration of stem cells is included in the composition so as to provide a reparative effect on the user's sexual organs.

Thus, the composition of the present invention comprises therapeutically effective amounts of a human sex hormone and hyaluronic acid. The composition may further include a therapeutically effective amount of a pheromone in some embodiments. In other embodiments, the composition further comprises stem cells. The composition is preferably provided as a cream for topical application directly to the sex organ or the user, such as the user's vagina. In this way, the composition can treat the skin of the sex organ for repairing and rejuvenating the same. Further, the sex hormones can be absorbed through the skin into the body.

In order for the composition to be formed into a cream, water and any of various emulsifying agents are included in the composition. The emulsifying agent helps to bind the various components together. Further, the emulsifying agent provides a thickened cream that can be easily applied by the user onto a desired area. An effective amount of water may be used in order to dissolve or suspend the various components so as to form the cream as required.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A composition for improving libido, comprising:
a therapeutically effective amount of a hyaluronic acid and a human sex hormone selected from the group consisting of testosterone and estrogen.

2) The composition for improving libido of claim 1, further comprising a pheromone.

3) The composition for improving libido of claim 1, further comprising a therapeutically effective concentration of stem cells.

4) The composition for improving libido of claim 1, further comprising an emulsifying agent.

5) A topical composition for use on human sexual organs, consisting of:
a human sex hormone selected from the group consisting of testosterone and estrogen;
hyaluronic acid; and
a pheromone.
